(19) United States
(12) Patent Application Publication
(10) Pub. No.: US 2023/0225715 A1
(43) Pub. Date: Jul. 20, 2023

(54) SAMPLING DEVICE

(71) Applicant: Readygo Diagnostics Limited, North Wraxall, Chippenham, Wiltshire (GB)

(72) Inventor: Ben Cobb, North Wraxall (GB)

(21) Appl. No.: 17/918,388

(22) PCT Filed: Mar. 22, 2021
(86) PCT No.: PCT/EP2021/057315

§ 371 Date: Oct. 12, 2022

(30) Foreign Application Priority Data
Apr. 16, 2020 (GB) 2005566.1
Oct. 26, 2020 (GB) 2016949.6

(51) Int. Cl. A61B 10/00; B01L 3/00; C12Q 1/6844

(52) U.S. Cl. CPC A61B 10/0045; B01L 3/5023; C12Q 1/6844; B01L 2300/047; B01L 2300/0832; B01L 2300/0861; B01L 2300/123; B01L 2400/0481; B01L 2200/10; B01L 2200/16; B01L 2300/1816

(57) ABSTRACT

The device comprises a nib (12) having a working surface (16) exposed or exposable for acquiring a biological sample and a porous structure to absorb the sample thus acquired. A reservoir (28) provides fluid under pressure to the nib via a valve (29), conveying and dispensing the sample into a reaction chamber (14), where it reconstitutes a dried-down reagent (43) to perform an analytic reaction on the sample, for example, an isothermal amplification of nucleic acid released from the sample by a membrane-disrupting agent pre-functionalised in the porous nib. The nib may be initially mounted (A) in the outlet of the reservoir or (B) in the inlet to the reaction chamber, in either case with its working surface (16) initially exposed to acquire the sample before the components of the device are assembled for performing the analytical procedure.

### CROSS REFERENCE TO RELATED APPLICATIONS

This application is a U.S. National Stage Application filed under 35 U.S.C. § 371, based on International Patent Application No. PCT/EP2021/057315, filed on Mar. 22, 2021, which claims priority to UK Patent Application No. GB2005566.1, filed on Apr. 16, 2020, and UK Patent Application No. GB2016949.6, filed on Oct. 26, 2020. The entire contents of each of the above applications are incorporated herein by reference.

### FIELD OF THE INVENTION

The present invention relates to collecting clinical samples and preparing them for analysis, for example by molecular biology processing techniques such as nucleic acid amplification and/or detection; and more particularly to a sampling device for that purpose, especially useful in a clinical point of care (POC) or point of need (PON) setting, but also for use in sending to a remote laboratory for testing. It further envisages the device in the form of a multifunctional kit from which a purpose-specific embodiment can be assembled, suited to the investigation under consideration.

### BACKGROUND TO THE INVENTION

The Polymerase Chain Reaction (PCR) is a convenient method of amplifying nucleic acids, employed to test for the presence of specific nucleic acids (DNA or RNA) in a biological sample. It is used among other purposes as a diagnostic method to identify markers for pathogens or diseases. Other methods of amplifying nucleic acid samples include isothermal amplification methods such as Recombinase Polymerase Amplification (RPA) and Loop-Mediated Isothermal Amplification (LAMP).

Prior to any such diagnostic test there needs to be a certain amount of preparation of the sample in order to present nucleic acids in a state that is compatible with the amplification process being used. Laboratory-based extraction methods are generally geared towards providing high concentrations of high purity nucleic acid, the aim being to obtain as much nucleic acid as possible. This comes at the expense of simplicity, and lab-based extraction techniques often require access to reagents and equipment such as centrifuges that are not compatible with clinical settings.

Preparation of clinical samples requires just enough nucleic acid to perform the test. This is an important distinction because it offers significant simplification of the extraction process. It is known for example, that the use of a simple filter paper enables the capture of enough nucleic acid from a target in a biological sample for a subsequent diagnostic via PCR, even from complex sample matrices such as whole blood (Fuehrer et al. J Clin Microbiol. 2011, 49(4), 1628-1630; Bu et al. Analytical Biochemistry 375(2), 370-372; Zou et al (2017) Nucleic acid purification from plants, animals and microbes in under 30 seconds, PLoS Biol 15(11), e2003916).

### SUMMARY OF THE INVENTION

Described here is a disposable device for collection and processing of biological samples, particularly—but not exclusively—for the purpose of DNA and RNA analysis procedures. The device provides enough nucleic acid for a molecular diagnostic test. Although the device is primarily described herein in conjunction with use in molecular diagnostic testing for nucleic acids, it will be appreciated that other biomolecules may be collected and/or processed with the invention, for example, proteins, lipids etc; as may cell fragments such as cell membrane or cell coat fragments, or subcellular organelles.

The aim of the invention is to provide a configuration of the device that enables collection of different sample types which may exist in various physical forms such as liquids (e.g. blood, saliva or urine) or as biofilms or cellular material on the surface of tissues or objects (e.g. touch surface DNA samples in forensics).

Also described are optional modifications to the basic form of the device that enable concentration of components (eg, nucleic acids) from the sample in order to enhance sensitivity, or that enable larger volumes of sample to be obtained, or to be obtained more simply.

According to a first aspect of the present invention there is provided a device for obtaining biological samples for analysis, comprising:

(a) a nib having:
a working surface exposed or exposable for acquiring a biological sample, and also a porous structure suitable for the absorption of biological sample matter thus acquired, and for the passage of liquid through the nib;

the nib being connected or connectable to:

(b) a body having:
a conduit leading at one end to the nib and at the other end to a bulb manually operable to push liquid towards the nib and/or withdraw liquid from the nib.

A related second aspect of the invention provides a device for obtaining biological samples for analysis, comprising:
(a) a nib having a working surface exposed or exposable for acquiring a biological sample, and having also a porous structure suitable for the absorption of biological sample matter thus acquired;
(b) a body having a form suitable for holding in, and manipulation by, the hand, and wherein the nib is connected or connectable to the body; and
(c) a reservoir adapted for fluid communication with the nib to provide the passage of fluid through the nib.

The following remarks apply to each of these embodiments, unless otherwise noted.

The reservoir may be located within the body (for example, as the manually operable bulb of the first aspect), or may be located within a separate component of the device (for example, as the cap as will be described herein). The reservoir may be operable to push fluid towards the nib and/or to withdraw fluid from the nib. Preferably the reservoir is operable by manually squeezing the reservoir, although other options are available—for example, a plunger, a button, a lever, and the like. In some embodiments the reservoir is pressurised to provide fluid to the nib. The reservoir may be removably attached to the nib—this applies whether the reservoir is in the body (where the body and nib are removably attached), or as a separate cap (which can then be removably attached to the nib and/or body). This arrangement facilitates provision of a kit form for the device, since the various components may be interchanged or replaced if needed.

The device may further comprise a flow-control means operable to allow the flow of fluid between the reservoir and the nib; for example, a tap or pincer as described herein.

The reservoir may contain a liquid reagent formulation for treatment, preferably elution, of the sample obtained on the nib.

The body is preferably in a form suitable for holding in, and manipulation by, the hand. For example, the body may be generally elongate, and preferably has similar dimensions to a writing implement (such as a pen or pencil). In this way, the body and nib combination will feel familiar to a user, and can readily be applied to collection of samples. Whereas the nib is normally expected to be a disposable element, the body may be configured for re-use with new nibs.

The present invention provides a convenient means of obtaining and preparing a sample for analysis, particularly by nucleic acid amplification processes such as PCR, RPA or LAMP, but also potentially by other analytic approaches such as immunodection. This is achieved, at least in part, by the nib that is used to collect the sample by absorption of liquid or part-liquid, or to wipe a surface containing the biological sample.

In one form, the nib has a chisel- or slanted chisel-shaped working surface to facilitate acquisition of sample at a fine point, at a fine edge, or as a broad stroke. This may be particularly useful in forensics for obtaining samples from various surfaces.

Coupled to the nib by the conduit (where present), the squeezable bulb or the reservoir more generally in one mode of action provides a liquid to (and through) the nib, releasing analyte (e.g. nucleic acid) for analysis.

The body may be fitted with a tap or pincer that initially closes the conduit from the bulb to the nib, and is displaceable to open the conduit and allow the flow of liquid between the bulb and the nib. In other embodiments, no such closure is provided, and the conduit from the bulb to the nib is open. The liquid may be used in various ways; in one embodiment the liquid may be provided to the nib prior to sample collection in order to dampen the nib. This can facilitate sample collection from a dry sample surface (for example, forensic collection from areas which have been touched; or collection from the anterior nares). In other embodiments, the nib can be dry for collection eg of liquid samples such as blood, saliva, urine, etc, and the liquid in the bulb later used for retrieving analyte from the nib.

Note that certain embodiments of the invention need not supply liquid in a squeezable bulb as such. Other formats may be equally suitable for use with the invention; for example, a solid chamber incorporating a plunger, a button, or the like to allow for expulsion of the liquid under pressure. The plunger or button may be indexed to permit more accurate determination of the amount of liquid to be provided.

Suitably, at least one removable cap is provided to shroud the nib before and/or after use. One such cap may be provided over the nib prior to use, and is replaceable after use to protect the sample obtained on the nib.

A cap may be provided with an opening to allow sample-containing droplets to be dispensed under pressure from liquid squeezed from the bulb through the nib.

A reaction chamber—conveniently in the form of the removable cap—may be provided containing a reagent formulation for treatment of the sample obtained on the nib. The reagent formulation is suitably in a freeze-dried or dried-down form to provide room-temperature stability for long-term storage. Such reagents may, for example comprise a protease or detergent, or amplification reagents for the detection of specific nucleic acid targets from the sample (for example, by isothermal nucleic acid amplification, preferably using one of the methods recited in Table 1). In alternative embodiments, the reaction chamber and reagent formulation may be provided within the body; for example, in a chamber located between the bulb and the nib. In such embodiments, in use liquid may be provided to the nib from the bulb and subsequently withdrawn into the chamber in order to carry sample into the chamber where the reagents may act. The cap (and/or the chamber where present) may be optically transparent, to permit a user to see the presence of the sample and/or reagents. In some embodiments, the reagents may include means to provide a colour-change reaction under defined conditions (eg, the presence of a particular target in the sample), thereby providing a rapid and easy readout of a sample analysis. In a further variation of the invention, the reagents may be provided within the body, and the cap may include a liquid which may be provided through the nib to the body.

Suitably, the cap is initially fitted with a seal over the reagent, which seal can be removed, broken, or opened to expose the reagent to sample carried by liquid squeezed from the bulb through the nib. The seal may take the form of a duckbill valve, which allows fluid flow from the nib to the reagent, but not in the reverse direction. However, a valve for that purpose may not be necessary, as described below in a novel example of use of the invention.

The nib may be provided with a hydrochromic mark to indicate the quantity of aqueous sample acquired. For example, the mark may be provided at a certain point along the length of the nib, so as to indicate when a predetermined amount of aqueous sample has been drawn into the nib. Alternatively, the mark may take the form of a water-sensitive dye throughout the nib which either appears or disappears in proportion to the amount of water present.

Means may be provided to facilitate heating of the nib for drying the sample, for concentrating the sample, or for increasing the volume of sample that can be drawn up. This heating means preferably comprises an element capable of being heated by induction. The nib may include a thermochromic mark to indicate that an appropriate temperature range has been achieved.

The present invention provides a convenient means of obtaining and preparing a sample for analysis (for example, by nucleic acid amplification processes such as PCR or isothermal amplification). This is achieved, in part by the nib that is used to collect the sample by absorption of liquid or part-liquid, or to wipe a surface containing the biological sample. Coupled to this nib through a fluid connection is a squeezable container (or a reservoir which is otherwise operable) that, in one mode of action, provides a liquid to (and through) the nib, releasing analyte (e.g. nucleic acid) for analysis.

As described in more detail below, the nib may comprise an active agent for treatment of the sample obtained on the nib, preferably the nib being functionalised with said active agent. The active agent preferably comprises one or more membrane-disrupting reagents that have the ability to lyse cells (eg bacteria) or viruses, releasing components thereof. For example, the DNA and/or RNA of lysed cells may be collected for further processing or analysis; alternatively, lipids, proteins or peptides may be collected, or subcellular organelles, or cellular fragments including cell membrane or cell wall fragments. Preferably the active agent is non-toxic to humans. One preferred active agent comprises a quaternary ammonium compound (QAC), and a more preferable active agent is cetyl pyridinium chloride, although alternatives (for example, as recited in Table 2, either individually or in combination) may be used. In some embodiments the active agent is in lyophilised or dried-down form, and the reservoir contains aqueous fluid for re-hydrating the active agent. Other uses for the active agent may be to capture certain components that may be inhibitory to the analysis process.

An optional modification of the device enables concentration of components (for example, nucleic acids) from the sample in order to enhance sensitivity or that enables larger volumes of sample to be processed.

Thus, the invention can provide a single disposable device to obtain a sample, and test for the presence of specific target analyte, such as nucleic acids, in the sample.

In some embodiments of the device, (a) the nib is located in an outlet from the reservoir, from which it projects for sample collection, and for subsequent connection to an inlet of the reaction chamber; or (b) the nib is located in an inlet to the reaction chamber, from which it projects for sample collection, and for subsequent connection to an outlet from the reservoir.

Preferably the nib is removably connectable to the body, and the device is provided with a plurality of replaceable nibs and/or a plurality of bodies and/or a plurality of reaction chambers.

According to another aspect of the present invention there is provided a kit for assembling a sampling device as described here, the kit comprising a selection of nibs and/or bodies, and/or accessory elements such as caps and reagents, from which a sampling device of the present invention can be assembled suited to a particular intended use. This is important where the nature and characteristics of the target analyte are not known, but also where the market for the sampling device is very specific or restricted—e.g. in forensic examination of surfaces.

Although mainly described herein in relation to obtaining and using test samples for analysis of their characteristic nucleic acid content, the present invention can also be applied to immunological (antibody/antigen) testing procedures, or indeed to collection and/or detection of other analytes which may be found in a biological sample where suitable detection reagents exist. For example, lipids, proteins, peptides, subcellular organelles or fragments, cell membrane or cell wall fragments, etc, may all be collected and processed in suitable applications of the invention.

The present invention is able to provide a test sampling device and method for its use, in which the device comprises a porous matrix to absorb test sample in a defined volume, and to expose the sample to an active ingredient that has been pre-functionalised into the nib through a drying process. The sample rehydrates those components as it wicks into the nib. From there, the extracted nucleic acids (or proteins in the case of immunodiagnostics, or lipids, etc) are available immediately, or can dry down during shipping for subsequent elution through buffer exchange by passing buffer back through the nib.

A further aspect of the present invention thus provides a method for collecting biological samples for analysis, the method comprising:
applying a nib having a porous structure suitable for the absorption of biological sample to a surface having a biological sample thereon;
allowing the biological sample to absorb into said nib; and
passing a fluid through said nib in order to wash absorbed biological sample from said nib into a reaction or collection chamber.

The nib may comprise an active agent for treatment of the sample obtained on the nib, preferably the nib being functionalised with said active agent. The active agent preferably comprises one or more membrane-disrupting reagents that have the ability to lyse cells (eg bacteria) or viruses, releasing components thereof. For example, the the DNA and/or RNA of lysed cells may be collected for further processing or analysis; alternatively, lipids, proteins or peptides may be collected, or subcellular organelles, or cellular fragments including cell membrane or cell wall fragments. Preferably the active agent is non-toxic to humans. One preferred active agent comprises a quaternary ammonium compound (QAC), and a more preferable active agent is cetyl pyridinium chloride, although alternatives (for example, as recited in Table 2, either individually or in combination) may be used. In some embodiments the active agent is in lyophilised or dried-down form, and the fluid passed through the nib re-hydrates the active agent. In some embodiments, the method comprises passing an initial flow of fluid through the nib (eg, to rehydrate the active agent, and/or to moisten the nib) prior to allowing the biological sample to absorb into said nib, and the wash step takes place with a subsequent flow of fluid through the nib.

### DRAWINGS AND EXEMPLIFICATION

FIG. 1 is a plan view of a device for sample collection and preparation;

FIG. 2 shows an exploded plan view of the principal components of the device

FIG. 3 shows a plan view of the components of the handle, apart from

FIG. 4 which shows a removable pincer clip for shutting of the flow of liquid to and/or from the handle;

FIG. 5 shows an exploded plan view of the nib and its associated shaft and removable cap;

FIGS. 6, 7 and 8 show respectively plan, side and perspective views of a particular form of nib;

FIG. 9 shows diagrammatically a novel application of an embodiment of the present invention; and

FIG. 10 shows simplified illustrations of two variant devices.

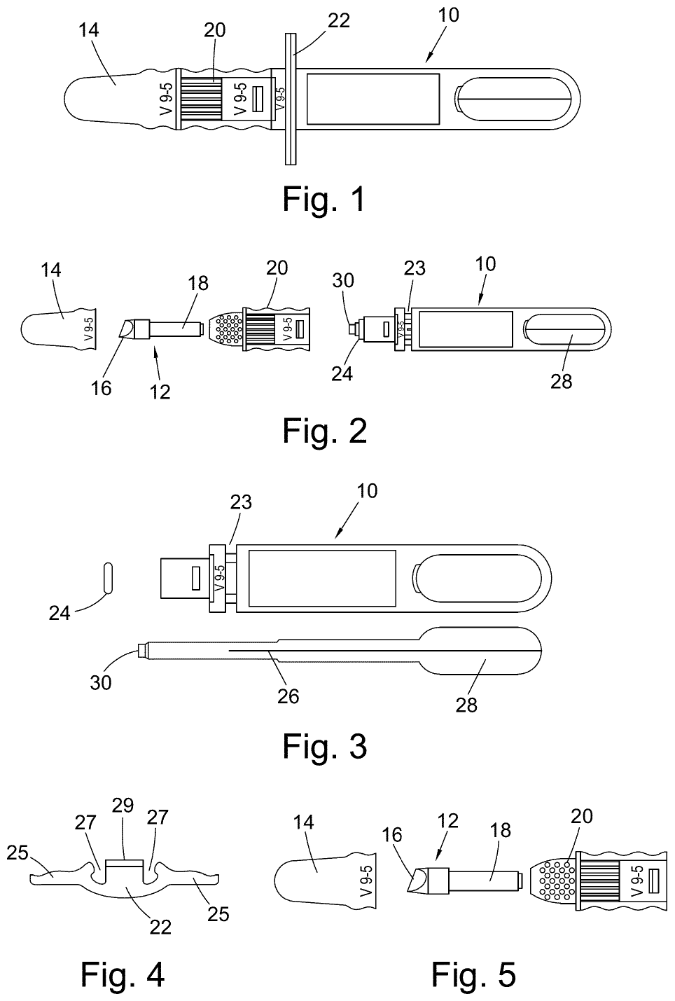

As shown in FIGS. 1 to 5, the device has the general form and size of a pen, comprising an elongate handle 10 having a sample collection nib 12 at one end covered by a removable protective cap 14.

As shown in more detail in FIGS. 2 to 8, the nib 12 is carried at the end of a shaft 18, and has a sampling end surface 16 which in this embodiment has a slanting chisel form so as to present a point 17, and an edge 19 flanked by flat surfaces 21.

The shaft 18 is held in a plastics collector component 20 suitably having external mouldings to assist the operator's grip.

The handle 10 contains an internal conduit 26 of flexible material extending lengthwise from an externally exposed squeezable bulb 28 to a stub shaft 30, onto which the collector component 20 is a push fit, clipped in place, and sealed by an O-ring 24 so as to be in fluid connection with the end of nib shaft 18.

A pincer clip 22, as depicted in more detail in FIG. 4, is displaceably fitted in a recess 23 on the handle to control the flow of liquid along the conduit 26. The pincer has the form of a plate with a pair of projecting arms 25 for manipulating the pincer, a pair of rebates 27 to releasably grip side portions of the handle, and a central plate 29 to bear upon the conduit and close it to the flow of liquid when the pincer is fully engaged in the handle 10. Note that in some embodiments the clip 22 need not be present at all; the flow of fluid from the bulb 28 through the conduit 26 may be controlled simply by making the conduit narrow enough to resist fluid flow in the absence of pressure exerted on the bulb 28, or by provision of a valve between the bulb 28 and conduit 26.

The nib 12 is of porous material to facilitate absorption of sample material, and the passage thereto of fluid squeezed from the bulb 28 and/or the passage therefrom of fluid to the bulb 28.

Optional and Operational Features

The sampling device of the present invention can be used just to take a sample and hold it for testing later or elsewhere, or it can provide means for performing such testing in the sampling device itself, for example, by LAMP, RPA, or other isothermal amplification methods (see e.g. appended TABLE 1). This would constitute a particularly advantageous use of the device.

The Nib

Suitable nib materials include cotton-based materials, or preferably a plastic based matrix such as PE or PVDF which can be manufactured in a range of densities to absorb and retain biological specimens of different viscosities. The precise detail of the nib construction may vary depending on the specific application for use of the invention, and the skilled person will be able to select appropriate materials and densities. For example, where the sample to be collected is saliva, one such suitable material is a cylindrical PE/PP wick of ˜90% porosity.

Marker pen nibs are often made of porous, pressed fibres such as felt or cellulose, or of porous, pressed plastic spheres, to create an open pore structure. The nib in the present invention may suitably be of similar material or structure, and preferably has a defined porosity (void fraction).

The nib can be designed to be task-specific or designed to cope with a range of different tasks. For forensics work it is convenient to have a nib design that allows fine swabbing of a surface for some applications, or a broad surface for sampling a wider area. It may also be convenient to provide a nib which may be used when damp, to collect dry samples for example from surface swabbing or samples from the anterior nares or the epidermis; or to provide a nib which may be used when dry, to collect liquid samples such as blood, urine, saliva, etc.

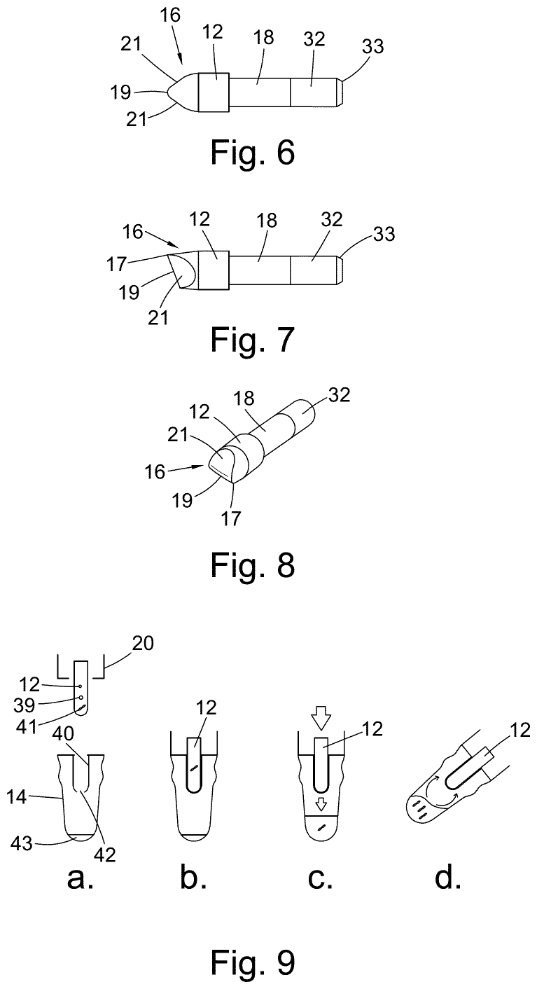

Felt pen nibs are often designed to allow ink strokes that are either fine or broad from the same single nib. The same design principles are employed here in the nib 12 as described and illustrated in FIGS. 6-8 of the drawings. The shape presenting the surfaces 16, 17, 19 and 21 is designed especially for collecting sample from a surface, for example in forensics work, where the slanting chisel form of the end surface 16 enables samples to be taken from a surface as a fine point, a fine edge, or as a broad stroke. However, the nib need not have the structural features described and illustrated there. At its simplest it may present merely a blunt or rounded end which is essentially a mere extension of the shaft 18 (as suggested diagrammatically in FIG. 9).

The porous form of the nib will provide capillary spaces for absorption of liquid test sample. The porous structure can be designed to draw a known volume of sample fluid, e.g. saliva, into its capillary spaces, thereby regulating the amount of sample taken and making the test more consistent.

The nib may have been treated to form a negatively charged surface and/or treated with an anionic detergent and/or treated with a biocidal agent to provide a means of lysing or removing cellular or viral components of the sample from the target nucleic acid components.

Cellulose fibres have a slight negative (anionic) charge. The hydroxyl groups (—OH) of cellulose can also be partially or fully reacted with various reagents to afford derivatives with useful properties. The nib may thus be provided with a negatively charged surface which binds and retains positively charged ions (such as Fe³⁺) from the test sample, and the bulb used to flow neutral or negatively charged ions and molecules (such as DNA) off the nib and away from cationic components.

Most living cells such as bacteria are surrounded by a fatty lipid bilayer. Lipid composition is not the same across subcellular membranes—mammalian plasma membranes having higher cholesterol and sphingolipid content. Viruses also have envelope lipids which are considered to be the same as the host membranes (phospholipids, sphingolipids, some cholesterol).

There are a number of different chemical groups that destroy the lipid layer of cells and consequently display antibacterial or antiviral properties. If these are used to functionalise the nib of the device, then cells (including bacteria) or viruses that soak into the nib will burst, releasing their DNA and RNA (or other cellular or viral components, such as lipids, proteins, subcellular organelles or fragments, membrane or cell wall fragments, etc).

TABLE 2, appended below, gives examples for functionalising the nib. The mouthwash formulations (shown in the shaded areas) seem particularly useful as they are known to be biosafe and effective, allowing the consumer to place the device into their mouth for self-collection of saliva for testing. For example, cetyl pyridinium chloride (CPC) is the active ingredient of various mouthwashes and has been shown to be a membrane-disrupting agent (Popkin et al, Cetylpyridinium chloride (CPC) exhibits potent, rapid activity against influenza viruses in vitro and in vivo, Pathogens and Immunity, 2017; 2(2):253-69).

Combinations may provide either targeted activity against the lipid bilayer of different organisms so that the same formulation can be used across a range of targets, or it may provide both a targeted preparation with additional antibacterial or antiviral activity to make the sample biosafe after use.

Reagents may be covalently bound to the nib by suitable chemical linkages through functionalisation (e.g. —OH groups), or they may be dried into the nib material and become active on hydration with the sample.

Drying down the formulation onto the nib, minimises dilution of the saliva (or other test sample) and therefore absorption of cleaved material back into the nib matrix. Mouthwash formulations indicated in Table 2 have been shown to act on viruses and bacteria in a matter of seconds. The nib therefore provides the following support functions:

1. A matrix on which to optionally dry down the antiviral and/or antimicrobial formulation, removing the need to dilute the saliva sample with the treatment;

2. Elicit saliva production when inserted into the mouth;

3. Absorb saliva that is elicited;

4. Contain a known volume of saliva.

A saliva sample may be collected and introduced into a tube for subsequent treatment. Or it may be desired to insert the sampler (nib) in the mouth and collect saliva in situ. In that case, the nib may be treated with compounds that have the ability to break open cells (eg bacteria) or viruses, releasing the DNA and/or RNA and/or other cellular or viral components, but are non-toxic and therefore make the product safe to use in that fashion,

The Squeezable Bulb

One use of the bulb is as a source of aqueous medium to wash test sample material from the nib and into the cap or other receptacle. It may also have the function of rehydrating dried-down reagent in the cap or in another location within the device.

Suitable liquids that might be held in the bulb include, purified water, milli-q water and buffer (e.g. TRIS-CI/TE buffer).

An initially empty bulb may also be required for use with a sample that has been dried (see below). With the pincer released, the bulb can be used to rehydrate the sample by drawing water or another carrier liquid up through the nib.

It is possible for the device of the present invention to be used without employing the squeezable bulb. For example, in forensics work the users may sometimes want to use their own water, rather than water supplied with the device, in which case the bulb could be supplied empty, or else the pincer left in place, or else the entire handle structure omitted, leaving just the nib and cap assembly for subsequent analysis of the test material captured in the nib. In further embodiments the bulb need not be squeezable, and other means may be provided for pushing fluid from the bulb to and/or from the nib (for example, a plunger, a button, a lever, and so on).

The Cap

As a closed structure, the cap 14 will hermetically seal the nib for safe transport.

Once nucleic acids or other target sample substances are extracted into the nib, they can be deposited into the cap or into test tubes, using liquid supplied from the squeezable bulb. However, variations in cap design, or the provision of one or more additional caps, may provide variable or extended, uses of the device.

For example, one design of cap could have a depositing aperture. Applying liquid under pressure from the squeezable bulb 28 would force liquid through the nib and into the cap. Continuing to apply pressure will force liquid through the aperture in the cap and allow a single drop of extracted material to be deposited into a tube such as a pipettor. Droplet size and size distribution is largely a function of aperture geometry.

In another example, a cap may be provided with reagent in a suitable form, for example as a freeze-dried or dried down pellet or coating on the inside surface of the cap, for performing a specific test on the sample. The reagent would be initially sealed in the cap, and the seal removed or punctured to allow the test to be performed when this cap is fitted to the handle in place of the original cap (or indeed this may be the original cap).

As the bulb is squeezed forcing liquid through the nib, nucleic acids from the test sample are collected by the liquid phase and washed into the cap, rehydrating the dried reagents. A test signal (e.g. colorimetric or fluorescent) may be thereby produced in the cap, and can be viewed from outside if the cap is transparent, e.g. of polycarbonate material.

In a variant of the device, dried reagent may be provided within the body, for example, in a chamber between the bulb and the nib. Fluid from the bulb can be used to rehydrate the reagent in the chamber, and either push the rehydrated reagent through the nib into the cap, or enter the nib and be drawn back into the chamber carrying the sample, where the reaction can take place.

Sample Drying, and Optionally Heating

The nib carrying the biological sample will dry naturally over time, determined by the external environment, temperature and humidity. This can be a convenient process if the sample is first collected and sent elsewhere for processing at some point in the future (e.g. next working day). However, natural drying times may be compromised by environmental factors (e.g. low temperature or high humidity), and there may be certain applications where it is preferable for drying to be done more rapidly to facilitate point of care (POC), point of need (PON) or on-the-spot testing.

Drying can be assisted by applying heat to the device, for example by placing the device into a hot space such as a drying oven or hot block. Drying ovens are often not convenient to use, however, because they represent a large dedicated piece of equipment which is not generally transportable outside of a laboratory. So heat may be supplied using a heating element associated with the structure of the device. For example, as shown in FIGS. 6-8 of the drawings, a collar 32 around the shaft 18 of the nib can be heated, preferably by a non-contact induction field. The induction heater could be supplied as a separate small device which would be portable and convenient for limited resource settings such as outside of the laboratory or within a small laboratory environment.

The heating element 32 may extend along the length of the shaft 18, or it may be positioned towards the end of the shaft distal from the nib as shown in the drawings. Any suitable induction material may be used, and may be a non-metal, such as carbon fibre.

Heating can also be used to thermally lyse the biological material in the sample contained in the nib. This may be of importance to release nucleic acids so that they are accessible to molecular amplification, and to render the sample in the device biologically safe.

Reagents may be developed by heating in a controlled manner according to the type of test being performed.

Sample Concentration

Apart from heating the nib to dry the sample, heat may be used to concentrate the sample, or to increase the volume of sample that can be drawn up using evaporation.

With the heating element 32 positioned as shown in the drawings, the heating is concentrated towards the distal end of the shaft 18, so that heating the shaft creates a thermal gradient with an evaporation front at the end 33 of the shaft.

Liquid in the nib is drawn up to the heat front via evaporation of the liquid phase at the end 33. This front will therefore create a continuous capillary draw of sample from the wetter nib. If the non-heated nib sits in a volume of liquid, for example in a reservoir, then as liquid is evaporated at the heated end 33, all the liquid will finally be drawn into the nib. In this way liquid in the nib will become concentrated until the supply of liquid sample is exhausted. This approach could also be used to draw a substantial sample of saliva by placing the nib under the tongue and applying the induction heating. Evaporated water is lost to the local environment as vapour (steam). Because volumes are small (<200 μL) and evaporated over a few minutes there is minimal condensate.

Heating can therefore be used not only to speed the drying process, but also to allow more sample to flow into the nib, thereby concentrating the analyte from a quantity of liquid larger than the volume within the nib itself.

Sample Release

The nib may need to be dried before releasing the extracted materials (see above).

Nucleic acids are negatively charged molecules. If the nib is also negatively charged then nucleic acids are repelled by the same charge and easily released into a mobile phase as it is forced through the nib.

Depending on the surface chemistry of the nib, the nib may also be positively charged in order to bind nucleic acids. This charge may be altered (e.g. by buffer exchange) to facilitate release of bound nucleic acids in the mobile phase. Often a change in pH will release certain charged molecules.

Other cellular or viral components may of course be collected; suitable chemistries and methods are known for permitting sample retention and subsequent release depending on the nature of the component and the nib.

Feedback on Use

The nib can be treated to provide feedback information on use. For example, indicator dyes can be employed on the nib to show when and/or how the nib has been used to obtain a liquid sample.

Thermochromic inks change colour when they have been exposed to certain temperatures. Hydrochromic inks change colour when wet. It may be desirable to use these in certain scenarios to indicate when the device has been used, and if the device has been used correctly. For example, these can be applied to the nib to provide a visual indication that the nib has been exposed to enough liquid, or if the nib has been heated up to for example >90° C.

Thus, by treating a distal part of the nib with a hydrochromic ink, colour changes will occur once the nib has received enough liquid to fill the nib to that marker position. This can be useful to give a positive indication that the device has been used, and that enough material has been collected.

The porous structure of the nib can be designed to draw a known volume of sample fluid, e.g. saliva, into its capillary spaces, thereby regulating the amount of sample taken and making the test more consistent.

Surface treatment chemistries can add lysing capabilities to the nib. Some biocidal chemistries by virtue of their mode of action also provide lysing capabilities. Other chemistries offer a means of capturing certain chemical groups which can be important for samples containing high concentrations of inhibitory components such as urine.

Lysing Chemistry

International patent applications WO00/62023 and WO02/16383 to Whatman, Inc, describe methods for lysing cells and isolating nucleic acids using coated filter medium (FTA-treated nitrocellulose), which is coated with an anionic detergent, for example SDS. The coating lyses the cells, and the FTA-treated filter adsorbs nucleic acids. The coating is not covalently bound to the filter and may be removed by washing. Nucleic acids can be eluted from the filter for subsequent processing. These materials may be useful in the practice of the present invention.

Biocidal Agents

European Patent application EP2855677A1 describes a paper-based system that works at room temperature, is capable of rupturing bacterial, fungal and viral cells, and releases nucleic acids into solution for direct-to-PCR analysis by functionalising the surface with a biocidal agent. The biocidal agent preferably comprises multiple functional groups. The functional groups preferably include a binding component which is involved in binding the agent to the substrate; a hydrophobic component; and a charged component. The hydrophobic component can interact with and penetrate the cell wall or cell membrane. These materials may be useful in the practice of the present invention.

In preferred embodiments, the hydrophobic component may be an alkyl chain, for example C5-C30 alkyl, preferably C10-C20 alkyl. As the alkyl chain penetrates the delicate cell wall, the wall is weakened and punctured. The charged component is preferably positively charged, and can attract a charged cell wall, and can disrupt ion flow and homeostasis on contacting a cell membrane, thereby helping to disrupt the cell and release the nucleic acids. The charged component is preferably a quaternary ammonium group. The binding component may comprise a hydroxyl group.

In preferred embodiments, the functional groups are preferably an alkyl chain (the hydrophobic component), a silyl group (the binding component), and an ammonium chloride group (the charged component). Preferred biocidal agents include silylated quaternary ammonium compounds (SiQACs); in particular 3-(trimethoxysilyl) propyldimethyloctadecyl ammonium chloride (3-TPAC). Other biocidal agents include benzyl ammonium chlorides. The lethal mode of action of SiQACs is generally accepted to proceed by adsorption of the positively charged molecule onto the negatively charged cell surface, disruption of the cell membrane by a lipophilic chain on the SiQAC molecule, and diffusion through the membrane leading to cell lysis.

The skilled person will be aware of other suitable biocidal agents that may be used. The selection of a particular agent will be guided by the presence of the preferred functional groups described above, and the nature of the intended biological sample—for example, where the sample to be processed is a mammalian cellular sample, then there is no cell wall to penetrate, and other functional groups may be appropriate.

For a review of other compositions which may be used, see “Antimicrobial Polymers in Solution and on Surfaces” (Siedenbiedel and Tiller (2012) Polymers, (4) 46-71). Specific examples of biocidal agents which may be used in the present invention include:

(i) Telechelic poly-(2-alkyl-1, 3-oxazolines).

(ii) Cellulose based fibres with an antimicrobial DDA group grafted via PEtOx, which kills approaching microbial cells on contact (Bieser et al., Contact-Active Antimicrobial and Potentially Self-Polishing Coatings Based on Cellulose, 2011, Macromol. Biosci., 11, 111-121).

(iii) Saponins (steroid or triterpenoid glycosides), common in a large number of plants, and have long been known to have a lytic action on erythrocyte membrane, many saponins being known to be antimicrobial (Francis et al., British Journal of Nutrition. 2002, (88) 587-605). Extensive research has been carried out into the membrane-permeabilising properties of saponins. These structurally diverse compounds have also been observed to kill protozoans and to act as anti-fungal and antiviral agents. Isolated cell membranes from human erythrocytes when treated with saponin developed pores of 40-50 Å diameter, as against the 80 Å pores produced in artificial membranes (Seeman et al. Structure of membrane holes in osmotic and saponin hemolysis, 1973, Journal of Cell Biology (56), 519-527).

Binding Components

Chitosan-modified Fusion 5 filter paper (unmodified ones purchased from GE Healthcare) have been successfully developed for DNA extraction and concentration (Francis et al., British Journal of Nutrition, 2002, (88) 587-605). The modified filter paper employs two separate mechanisms: the physical entanglement of long-chain DNA molecules with the fibre matrix of the filter paper, and the electrostatic adsorption of DNA to the chitosan-modified filter fibres. This enables a bonding and capture of DNA to the fibres and subsequent washing of inhibitors prior to PCR.

Reservoir Variants

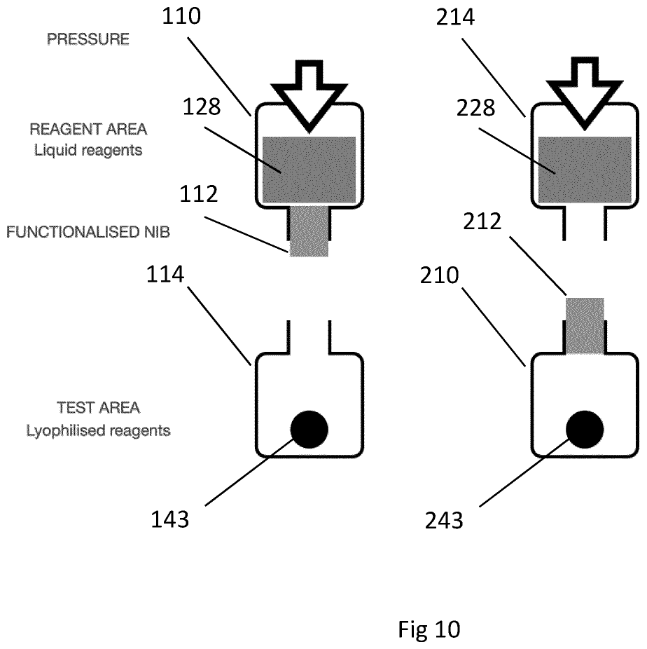

Two simplified illustrations of variant reservoir arrangements are shown in FIG. 10. In the left hand embodiment, the handle 110 includes a nib 112 and a reservoir 128 containing liquid reagents (eg, Tris-HCl buffer) which can be placed under pressure, for example, by squeezing the handle as described herein. The cap 114 includes dehydrated or lyophilised reagents 143. When the cap 114 is placed over the nib 112 and the reservoir 128 placed under pressure, liquid is forced through the nib 112, where the sample is eluted, and into the cap 114 so reconstituting the reagents 143 and allowing the detection reaction to take place.

In the right hand embodiment, the handle 210 contains the lyophilised or dehydrated reagents 243, while the cap 214 includes the reservoir 228. When the cap 214 is placed over the nib 212 and pressurised, the liquid flows through the nib 212 into the reagent compartment of the handle 210.

In some embodiments, the reservoir 128, 228 may contain the liquid under pressure, and be sealed by, eg, a valve or a membrane which can be opened or pierced once the cap is placed over the nib, to allow fluid flow. Where the reservoir is in the cap, the valve may be formed by a moulded portion comprising multiple arms or segmentswhich fit around the nib. When the cap is placed on the device, the arms of the moulded portion are urged apart by the nib, thereby opening the valve in the cap and allowing the liquid to flow from the cap through the nib. Where the reservoir is in the body 110, the cap may be designed to urge the nib 112 into the body so as to open an equivalent valve provided in the body, or so as to pierce a sealing membrane.

Use Case Scenarios

1. Sample Collection

The sample collector can be used on its own as part of a large scale or centralised testing process. For example, the 2020 SARS-CoV-2 pandemic has seen various mass testing strategies implemented worldwide. These tests have generally been done within a centralised laboratory network, processing hundreds of thousands of samples each day.

Using the sample collector with a nib that has been functionalised with compounds known to break open the lipid capsid of the virus, it is possible to collect samples at home for subsequent posting back through the post. This reduces the need to take nasopharyngeal samples which requires a skilled person to take effectively. The sample collector may be provided with a gasket part that interfaces with the plastic pipette tips of automated liquid handling units in robotic sample processing laboratories. In some embodiments, the cap may be designed to receive a collected sample which is subsequently eluted from the nib using liquid from the bulb; the cap can then be removed, sealed, and sent for further processing. Alternatively, the nib itself may be removed and sent for further processing without elution.

The additional benefit is that virus is broken up and the RNA released into the tip as the sample is handled and transported back to the test facility. Elution of pre-lysed material back at the lab allows a simpler test of the sample without the need for extraction in the lab.

This same approach may be used for other targets which require more centralised testing.

The nib may be of different materials which offer different characteristics according to task. There are two main plastics that are especially useful, polyvinylidene fluoride (PVDF) and polyethylene (PE).

PVDF is a highly non-reactive thermoplastic fluoropolymer produced by the polymerization of vinylidene difluoride. PVDF is a specialty plastic used in applications requiring the highest purity, as well as resistance to solvents, acids and hydrocarbons. It is normally manufactured into nibs for pens which are made using the sintering process. They generally form nibs that have good flow characteristics and can be of differing ‘hardness’. This is especially useful if the nib is required to be used in a physical process such as wiping a surface or crushing a sample in order to release an exudate to be then drawn into the nib and tested.

PE is a lightweight, durable thermoplastic with variable crystalline structure. It is one of the most widely produced plastics in the world (tens of millions of tons are produced worldwide each year). PE is used in applications such as films, tubes, plastic parts, laminates, etc. PE has been used to create materials that have an open structure that is particularly useful in collection of saliva. The manufacture can be controlled to provide materials within different retention and flow rates.

2. Sample Collection and Dispense

This combination allows sample collection and elution of extracted material into another test such as a molecular diagnostic or an immunodiagnostic. A known volume is drawn into the nib material where the specific functionalisation acts on microorganisms contained within the sample matrix. The extracted material is washed off the tip using the reagent contained in the dispenser/bulb element of the device. Pushing the rehydration buffer from the dispenser through the nib washes the saliva into the test cap. Control by volume creates a known dilution of the saliva since the nib absorbs a defined volume (e.g. 25 μL) and the dispenser/bulb has a known volume (e.g. 2004).

3. Sample Collection, Dispense and Test

This combination allows both professional point-of-care, and consumer over-the-counter testing. Collection of saliva by sucking on the end of the nib generates saliva which is collected into the nib. After 5 minutes sufficient saliva is collected, and the targeted bacteria are broken down by the antimicrobials functionalised into the nib matrix. This releases the nucleic acids into the aqueous phase. Pushing the rehydration buffer from the dispenser/bulb through the nib washes the saliva into the test cap. Control by volume creates a known dilution of the saliva since the nib absorbs a defined volume (e.g. 25 μL) and the dispenser/bulb has a known volume (e.g. 2004). Test chemicals, preferably for an isothermal amplification such as recombinase polymerase amplification, are rehydrated and over a period of time will develop a colour change if the target DNA is present in the nib. This may be of a simple colour change (e.g. yellow to red) using a pH indicator or may be development of a fluorescent colour (e.g. blue to green).

Some Specific Applications The applications of the present invention are broad since the device provides all of the steps required from sampling, sample processing, elution and testing. Examples of specific applications are given below;

Periodontal disease, or periodontitis, is caused by a dysbiosis within the dental plaque microbial community causing the disruption of tissue homeostasis (Hajishengallis et al, Beyond the red complex and into more complexity: the polymicrobial synergy and dysbiosis (PSD) model of periodontal disease etiology, Mol Oral Microbiol. 2012; 27:409-419). It affects 10-15% of adults and is the most common cause of tooth loss worldwide. A test that could be bought OTC would allow people to better track the abundance of bacteria causing periodontal disease at home.

Providing a system capable of reporting a positive if any of the main causes of periodontal disease is present in the sample would be of particular use as an OTC device—for example, the following six species may be considered indicative of periodontal disease:
Porphyromonas gingivalis, ATCC33277
Actinobacillus actinomycetemcomitans, ATCC29523
Fusobacterium nucleatum, No. 2
Tannerella forsythensis, ATCC43937
Prevotella intermedia, ATCC25611
Streptococcus anginosus, ATCC33397

Porphyromonas gingivalis and Actinobacillus actinomycetemcomitans have been shown to be key bacteria responsible causing a chronic inflammatory disease of the periodontium, the tissues that surround and support the teeth. Both species were found in 33% and 44%, respectively, of subjects with moderate periodontitis and in 60% and 40%, respectively, of the subjects with advanced periodontitis. (Troil-Lindén et al, (1995). Salivary Levels of Suspected Periodontal Pathogens in Relation to Periodontal Status and Treatment. Journal of Dental Research).

Some cancers: Around 1 in 4 mouth cancers and 1 in 3 throat cancers are HPV-related, but in younger patients most throat cancers are now HPV-related. A study carried out in 2009-10 concluded that 1 in 10 American men and less than 4 in 100 American women had HPV infection in the mouth. Another study published in 2017 found that in America, 6 in 100 men and 1 in 100 women carried potentially cancer-causing types of HPV in their mouth.

There is solid evidence to support the use of saliva as a subject for diagnostic testing for the detection of HPV DNA in oropharyngeal cancer (OPC) patients. (Rosenthal et al. Detection of HPV related oropharyngeal cancer in oral rinse specimens, Oncotarget 2017; 8:109393-109401 and Chai et al, A pilot study to compare the detection of HPV-16 biomarkers in salivary oral rinses with tumour p16(INK4a) expression in head and neck squamous cell carcinoma patients. BMC Cancer. 2016; 16:178).

Furthermore, the detection of HPV DNA in saliva collected by different methods (drool or oral rinse) yields comparable results and shows good sensitivity for the detection of HPV, again supporting the feasibility of using saliva as a diagnostic medium for OPC. (Tang et al, High-risk human papillomavirus detection in oropharyngeal cancers: Comparison of saliva sampling methods, Head Neck. 2018)

Providing a system capable of reporting a positive if any of the main cancer-causing genotypes of HPV were present in a saliva sample would be of particular use as an OTC device.

White spot disease in penaeid shrimp is caused by the white spot syndrome virus (WSSV). It is the most economically important disease of farmed warm-water shrimp, causing extensive economic losses estimated from $8 to $15 billion since its emergence in the 1990s. Early diagnosis of disease is critical in the management of outbreaks and to avoid crop losses.

Providing a system capable of first crushing a portion of shrimp to release the haem containing WSSV, then absorbing the haem into the nib for nucleic acid extraction, elution and test would be of particular use as a device for agricultural diagnostics.

A Novel Example of Use

Referring to FIG. 9 of the drawings, the nib 12, of suitable absorbent material—e.g. PE fibres or sintered PVDF—is shown diagrammatically as of cylindrical form projecting from the collector component 20, and terminating in a simple rounded end.

The nib has been pretreated with a cell-lysing functionalising substance, such as described in Table 2, preferably cetyl pyridinium chloride (CPC), which is now in dried form.

The cap 14 contains nucleic acid amplification reagents 43, dehydrated or lyophilised or dried down on the interior surface at the distal end of the cap. They contain test elements specific for the thing being tested for. Various amplification methods could be used, but isothermal amplification is preferred. In RPA (recombinase polymerase amplification) the primers and polymerases are dried down, and the buffer remains liquid to rehydrate those elements. This is a particularly useful configuration because RPA uses crowding agents such as PEG (polyethylene glycol) that does not like being dried down, and so can be kept separate. Use of a foam-like structure for the nib—particularly polyethylene (PE) or polyvinylidenefluoride (PVDF)—means that its porosity allows a PEG solution to pass through.

The cap 14 has an internal sleeve 40 to receive and closely surround the nib, apart from a small aperture 42 at the inner end of the sleeve.

As shown in step (a), the nib has been contacted with a biological sample to be tested, such as saliva, which has been absorbed into the nib. The dark spot 39 indicates the presence of intact target cell (virus, bacterium, etc); the squiggly line 41 indicates nucleic acid released by CPC lysis of the target cell.

Step (b) shows the nib fully inserted into the sleeve 40, so that only a small area at the end of the nib is exposed to the interior of the cap through the aperture 42.

In step (c), rehydration fluid is forced from the bulb (not shown) through the nib material, washing the test sample into the cap void beyond the sleeve 40, and into contact with the amplification agents 43.

Typically, there is sufficient void in the cap to accommodate 200 μL thus pushed into it. The geometry of the cap is such that even with agitation there is sufficient void that the liquid cannot come into significant contact with the nib. Moreover, there is only a very small amount of nib that is exposed through the aperture 42, and the nib material is of course wet from the rehydration fluid, thereby preventing any appreciable volume of liquid from going back into the nib, even with significant agitation (as indicated in (d) below). It should therefore not be necessary to have a distinct valve component controlling the flow of fluid—the nib itself effectively becomes the valve when it is pushed fully into the cap.

In step (d) the target nucleic acid is now in the liquid contained in the cap, where the nucleic acid amplification agents 43 on the inside of the cap are rehydrated and act to produce multiple copies of the target nucleic acid, if appropriate aided by shaking and/or heating. As can be seen, the configuration should allow shaking of the cap to aid the process with minimal contact of the liquids with the small exposed area at the end of the nib. The arrangement is therefore both simple and effective.

The entire assembly of cap and collector component can if desired be detached from the handle and sent away for analysis or further treatment of the contents.

An important feature of this aspect of the invention is that the cell disrupting agent CPC, among others, is commonly used in OTC oral disinfectants, such as Oral B™ and therefore the nib can be safely and easily held in the mouth to take a saliva sample, thereby making this a very suitable test for home use, and not requiring medical supervision.

TABLE 1
Summary of isothermal amplification methods

| | |
|---|---|
| NASBA | Nucleic acid sequence-based amplification is a method |
| | used to amplify RNA |
| LAMP | Loop-mediated isothermal amplification is a single tube |
| | technique for the amplification of DNA. It uses 4-6 |
| | primers, which form loop structures to facilitate |
| | subsequent rounds of amplification |
| HAD | Helicase-dependent amplification uses the double |
| | stranded DNA unwinding activity of a helicase |
| | to separate strands for in vitro DNA amplification at |
| | constant temperature |
| RCA | Rolling circle amplification starts from a circular DNA |
| | template and a short DNA or RNA primer to form a |
| | long single stranded molecule |
| MDA | Multiple displacement amplification is a technique that |
| | initiates when multiple random primers anneal to the |
| | DNA template and the polymerase amplifies DNA |
| | at constant temperature |
| WGA | When MDA is used to amplify DNA from a whole |
| | genome of a cell it is called whole genome |
| | amplification. (Other methods of WGA include |
| | MALBAC, LIANTI, DOP-PCR) |
| RPA | Recombinase polymerase amplification is a low |
| | temperature DNA and RNA amplification technique. |

TABLE 2

| | | Active Ingredient | |
|---|---|---|---|
| Group | Active ingredient | concentration | Reference |
| Monoterpenes | Citral | 25 μg/ml | Astani et al, Comparative study on the |
| Derived from | Eucalyptus oil | 100 μg/ml | antiviral activity of selected monoterpenes |
| Essential Oils | Tea tree oil | 7.5 μg/ml | derived from essential oils. Phytother Res. |
| Chlorhexidine | Chlorhexidine | 0.12% | 2010; 24(5): 673-679. |
| | | | Nazzaro et al. Effect of essential oils on |
| | | | pathogenic bacteria. Pharmaceuticals (Basel) |
| Saponins | triterpenoid saponin | | Simões et al, Mechanism of antiviral activity of |
| | | | triterpenoid saponins. Phytother Res. 1999 |
| | | | Jun; 13(4): 323-8 |
| | Polyphylla saponin I | 6.25-50 μg/mL | Pu et al, Polyphylla saponin I has antiviral activity |
| | | | against influenza A virus. Int J Clin Exp Med. |
| | | | 2015; 8(10): 18963-18971. |
| Povidone- | Povidone-iodine | 0.23%; (PVP-I: | Kariwa et al, Inactivation of SARS |
| iodine | | 0.47 and | coronavirus by means of povidone- |
| | | 0.23% w/v) | iodine, physical conditions and |
| | | | chemical reagents. Dermatology. |
| | | | 2006; 212 Suppl 1: 119-23 |
| | | | Meister et al, Virucidal Efficacy of Different |
| | | | Oral Rinses Against Severe Acute Respiratory |
| | | | Syndrome Coronavirus 2. J Infect Dis. |
| | | | 2020; 222(8): 1289-1292 |
| | | | Capriotti et al, A Novel Topical 2% |
| | | | Povidone-iodine Solution for the |
| | | | Treatment of Common Warts: A |
| | | | Randomized, Double-Blind, Vehicle- |
| | | | Controlled Trial. Dermatol Ther (Heidelb). |
| | | | 2015; 5(4): 247-252. |
| Quaternary | Cetylpyridinium | CPC is used in | Popkin et al, Cetylpyridinium Chloride (CPC) |
| ammonium | chloride (CPC) | medicated oral | Exhibits Potent, Rapid Activity Against |
| compounds | | rinses at | Influenza Viruses in vitro and in vivo. Pathog |
| | | concentrations | Immun. 2017; 2(2): 252-69 |
| | | 0.025-0.075% w/v | Baker et al, Repurposing Quaternary Ammonium |
| | | (250-750 μg/ml) in | Compounds as Potential Treatments for |
| | | the UK, while | COVID-19, Pharm Res (2020) 37: 104 |
| | | lozenges sold in | Schrank et al, Disinfectants, Effective |
| | | some countries | against Severe Acute Respiratory |
| | | contain 1.4-3.0 mg | Syndrome-Coronavirus-2? ACS Infect. |
| | | of CPC. >10 μg/ml | Dis. 2020, 6, 1553-1557 |
| | | shown to be | Kersting et al, Multiplex isothermal solid- |
| | | effective. | phase recombinase polymerase |
| | | | amplification for the specific and fast |
| | | | DNA-based detection of three bacterial |
| | | | pathogens, Microchim Acta (2014) |
| | | | 181: 1715-1723 |
| | | | O'Donnell et al, Potential role of oral rinses |
| | | | targeting the viral lipid envelope in SARS-CoV- |
| | | | 2 infection, Function, Volume 1, Issue 1, 2020 |
| | Benzalkonium chloride | | Rabenau et al, Efficacy of various disinfectants |
| | (BAC) | | against SARS coronavirus. J Hosp Infect. |
| | | | 2005; 61(2): 107-111 |
| | | | Ansaldi et al, (2004). SARS-CoV, |
| | | | influenza A and syncitial respiratory |
| | | | virus resistance against common |
| | | | disinfectants and ultraviolet |
| | | | irradiation Journal of Preventive |
| Mouth wash | Listerine Cool Mint- | | Meister et al, Virucidal Efficacy of Different |
| formulations | Ethanol, essential oils | | Oral Rinses Against Severe Acute Respiratory |
| | Dequonal- | | Syndrome Coronavirus 2. J Infect Dis. |
| | Dequalinium | | 2020; 222(8): 1289-1292 |
| | chloride (DAC), | | |
| | Benzalkonium | | |
| | cholride (BAC) | | |
| | Oral B Pro- | | |
| | Cetylpyridinium | | |
| | chloride (CPC) | | |
| | Crest Pro-Health Multi- | | |
| | Protection (C21H38ClN) | | |
| | mouthwash- | | |
| | cetylpyridinium | | |
| | chloride(C21H38ClN) | | |
| | BETADINE Gargle | | Martinez Lamas et al, (2020), Is povidone- |
| | and Mouth Wash | | iodine mouthwash effective against SARS- |
| | Povidone-iodine I | | CoV-2? First in vivo tests. Oral Disease |
| | (PVP-I) | | Cimolai, N. (2020). Efficacy of povidone- |
| | | | iodine to reduce viral load. Oral Diseases, |
| | | | https://doi.org/10.1111/odi.13557. |
| | | | Nakagawa et al, The efficacy of povidone- |
| | | | iodine products against periodontopathic |
| | | | bacteria. Dermatology. 2006; 212 Suppl |
| | | | 1: 109-11 |

1. A device for obtaining biological samples for analysis, comprising:
(a) a nib having a working surface exposed or exposable for acquiring a biological sample, and having also a porous structure suitable for the absorption of biological sample matter thus acquired;
(b) a body having a form suitable for holding in, and manipulation by, the hand, and wherein the nib is connected or connectable to the body; and
(c) a reservoir adapted for fluid communication with the nib to provide the passage of fluid through the nib.

2. A device according to claim 1 wherein the reservoir is located within the body.

3. A device according to claim 1 wherein the reservoir is operable to push fluid towards the nib and/or to withdraw fluid from the nib.

4. A device according to claim 1 wherein the reservoir is pressurised to provide fluid to the nib.

5. A device according to claim 1, wherein a flow-control means is operable to allow the flow of fluid between the reservoir and the nib.

6. A device according to claim 1, wherein the nib is associated with a hydrochromic mark to indicate the quantity of aqueous material acquired.

7. A device according to claim 1, wherein the reservoir contains a liquid reagent formulation for treatment, of the sample obtained on the nib.

8. A device according to claim 1, wherein the nib comprises an active agent for treatment of the sample obtained on the nib.

9. A device according to claim 8 wherein the active agent comprises one or more membrane-disrupting reagents that have the ability to lyse bacteria or viruses.

10. A device according to claim 8, wherein the active agent is non-toxic to humans.

11. A device according to claim 8, wherein the active agent comprises one or more agents selected from the group consisting of Citral, Eucalyptus oil, Tea tree oil, Chlorhexidine, triterpenoid saponin, Polyphylla saponin I, Povidone-iodine, Cetylpyridinium chloride (CPC), Benzalkonium chloride (BAC), Dequalinium chloride.

12. A device according to claim 8, wherein the active agent comprises cetyl pyridinium chloride.

13. A device according to claim 8, wherein said active agent is in lyophilised or dried-down form, and the reservoir contains aqueous fluid for re-hydrating the active agent.

14. A device according to claim 1 which includes means to facilitate heating of the nib or other component of the device.

15. A device according to claim 14, wherein the heating is facilitated by an induction-heater element.

16. A device according to claim 14, wherein a thermochromic mark is provided to indicate that an appropriate temperature range has been achieved.

17. A device according to claim 1, further comprising a reaction chamber to receive fluid supplied from the reservoir and discharged from the nib after contact with the sample.

18. A device according to claim 17, wherein the reaction chamber is located within the body.

19. A device according to claim 17, wherein the reaction chamber can be removably located so as to cover the nib.

20. A device according to claim 17, wherein the chamber has a transparent region for viewing the interior.

21. A device according to claim 17, comprising a reagent formulation for treatment of the sample in the reaction chamber.

22. A device according to claim 21, wherein the reagent formulation is in a lyophilised or dried-down form.

23. A device according to claim 21, wherein the reagent formulation comprises one or more reagents for the detection of specific nucleic acid targets in the sample.

24. A device according to claim 23, wherein said reagent formulation comprises reagents for isothermal amplification of nucleic acid.

25. A device according to claim 24, wherein the isothermal amplification is recombinase polymerase amplification.

26. A device according to claim 23 wherein the reagent formulation provides a visual signal in the event of detection of said specific nucleic acid targets.

27. A device according to claim 21 wherein the reagent formulation comprises one or more reagents for the detection of specific proteins, peptides, and/or lipids in the sample.

28. A device according to claim 27 wherein the reagent formulation comprises one or more reagents for immunoassay-based detection of a target in the sample.

29. A device according to claim 17, wherein:
(a) the nib is located in an outlet from the reservoir, from which it projects for sample collection, and for subsequent connection to an inlet of the reaction chamber; or
(b) the nib is located in an inlet to the reaction chamber, from which it projects for sample collection, and for subsequent connection to an outlet from the reservoir.

30. A device according to claim 1, wherein the nib is removably connectable to the body, and comprising a plurality of replaceable nibs.

31. A device according to claim 1, provided in kit form.

32. A method for collecting biological samples for analysis, the method comprising:
applying a nib having a porous structure suitable for the absorption of biological sample to a surface having a biological sample thereon;
allowing the biological sample to absorb into said nib; and
passing a fluid through said nib in order to wash absorbed biological sample from said nib into a reaction or collection chamber.

33. The method of claim 32 wherein the nib comprises an active agent for treatment of the sample obtained on the nib.

34. The method of claim 33 wherein the active agent comprises one or more membrane-disrupting reagents that have the ability to lyse cells or viruses, releasing components thereof.
